(19) United States
(12) Patent Application Publication
(10) Pub. No.: US 2026/0232781 A1
(43) Pub. Date: Aug. 13, 2026

(54) COMPOSITION FOR IMMUNIZING AGAINST PARASITE INFECTION AND METHODS USING THE SAME

(71) Applicant: YALE UNIVERSITY, New Haven, CT (US)

(72) Inventor: Choukri Ben Mamoun, New Haven, CT (US)

(21) Appl. No.: 19/155,901

(22) PCT Filed: Feb. 19, 2024
(86) PCT No.: PCT/US2024/016349

§ 371 Date: Aug. 12, 2025

Related U.S. Application Data
(60) Provisional application No. 63/485,960, filed on Feb. 20, 2023.

(51) Int. Cl. A61K 39/002; A61K 9/50; A61K 9/51; A61K 39/00; A61P 33/02

(52) U.S. Cl. CPC A61K 39/002; A61K 9/5068; A61K 9/5192; A61P 33/02; A61K 2039/55505; A61K 2039/55555; A61K 2039/55561; A61K 2039/55572; A61K 2039/55577; A61K 2039/575

(57) ABSTRACT

Described herein is a composition for immunization of a mammal against an infection by a parasite. The composition includes an extracellular vesicle produced by a red blood cell infected with the parasite; and a pharmaceutically acceptable carrier for parenteral administration. Also described herein is a method for immunizing a subject against an infection by a parasite. The method includes administering to the subject an effective amount of an extracellular vesicle exuded by the parasite, which resides with the host red blood cell.

### CROSS-REFERENCE TO RELATED APPLICATIONS

The present application claims priority under 35 U.S.C. § 119(e) to U.S. Provisional Patent Application No. 63/485,960, filed Feb. 20, 2023, which is incorporated herein by reference in its entirety.

### STATEMENT REGARDING FEDERALLY SPONSORED RESEARCH

This invention was made with government support under IR01AI152220-01 awarded by National Institutes of Health. The government has certain rights in the invention.

### BACKGROUND

Babesiosis and malaria are both zoonoses, i.e., infectious diseases that jump from a non-human animal to humans. The causal agents of Babesiosis are Babesia parasites and the causal agents of malaria are Plasmodium parasites—both of which invade and inhabit red blood cells of the hosts during part of their life cycles. Babesiosis is mostly transmitted to humans via tick bites while malaria is transmitted by mosquito bites.

Currently, there is no vaccine against human babesiosis and vaccines against malaria are limited in number and efficacy. There is a need for novel vaccines against these infectious diseases. The present invention addresses this need.

### SUMMARY

In some aspects, the present invention is directed to the following non-limiting embodiments:

### Composition

In some embodiments, the present invention is directed to a composition.

In some embodiments, the composition comprises an extracellular vesicle produced by a red blood cell infected with a parasite; and a pharmaceutically acceptable carrier for parenteral administration.

In some embodiments, when administered to a subject, the composition immunizes the subject against an infection of the parasite.

In some embodiments, the parasite is one or more Babesia parasites.

In some embodiments, the parasite is at least one selected from the group consisting of a B. duncani parasite, and a B. microti parasite.

In some embodiments, the composition further comprises an immunologic adjuvant.

In some embodiments, the immunologic adjuvant is at least one selected from the group consisting of an aluminum-based adjuvant, a lipid-based adjuvant, a nucleic acid-based adjuvant, and a saponin-based adjuvant.

In some embodiments, the extracellular vesicle is isolated by infecting the red blood cell cultured in a culture medium with the parasite.

In some embodiments, the extracellular vesicle is isolated by further centrifuging the culture medium at a centrifugation speed ranging from about 200 g to about 20,000 g and collecting at least a portion of the supernatant.

In some embodiments, the collected supernatant comprises the extracellular vesicle.

In some embodiments, the extracellular vesicle is isolated by infecting the red blood cell cultured in a culture medium with the parasite.

In some embodiments, the extracellular vesicle is isolated further by centrifuging the culture medium at a centrifugation speed ranging from about 200 g to about 20,000 g and collecting at least a portion of the supernatant.

In some embodiments, the extracellular vesicle is isolated further by centrifuging the supernatant at a centrifugation speed of about 80,000 g or higher so as to collect a pellet.

In some embodiments, the collected pellet comprises the extracellular vesicle.

In some embodiments, the extracellular vesicle is isolated by low-centrifugation speed isolation technique, such as a nano-particle based method, a polymer-based method, a filtration-based method, an immunoaffinity capture, a microfluidic isolation, a or size-exclusion chromatography.

### Method of Immunizing Subject

In some embodiments, the present invention is directed to a method of immunizing a subject against an infection by a parasite.

In some embodiments, the method comprises administering to the subject an extracellular vesicle produced by a red blood cell infected with the parasite.

In some embodiments, the parasite is one or more Babesia parasites.

In some embodiments, the parasite is at least one selected from the group consisting of a B. duncani parasite and a B. microti parasite.

In some embodiments, the subject is further administered with an immunologic adjuvant.

In some embodiments, the immunologic adjuvant is at least one selected from the group consisting of an aluminum-based adjuvant, a lipid-based adjuvant, a nucleic acid-based adjuvant, and a saponin-based adjuvant.

In some embodiments, the extracellular vesicle is an isolated extracellular vesicle.

In some embodiments, the extracellular vesicle is isolated by infecting the red blood cell cultured in a culture medium with the parasite.

In some embodiments, the extracellular vesicle is isolated further by centrifuging the culture medium at a centrifugation speed ranging from about 200 g to about 20,000 g and collecting at least a portion of the supernatant.

In some embodiments, the collected supernatant comprises the extracellular vesicle.

In some embodiments, the extracellular vesicle is isolated by the extracellular vesicle is an isolated extracellular vesicle, and wherein the extracellular vesicle is isolated by infecting the red blood cell cultured in a culture medium with the parasite.

In some embodiments, the extracellular vesicle is isolated further by centrifuging the culture medium at a centrifugation speed ranging from about 200 g to about 20,000 g so and collecting at least a portion of the supernatant.

In some embodiments, the extracellular vesicle is isolated further by centrifuging the supernatant at a centrifugation speed of about 80,000 g or higher so as to collect a pellet.

In some embodiments, the collected pellet comprises the extracellular vesicle.

In some embodiments, the subject is administered with a plurality of doses of the extracellular vesicle.

In some embodiments, the red blood cell is from the same species as that of the subject.

In some embodiments, the subject is a mammal.

In some embodiments, the subject is a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1 illustrates the timeline for a B. duncani immunization study using C3H/HeJ female mice, in accordance with some embodiments. Mice were immunized (1 prime and 2 boosts, two weeks apart) by the intramuscular (IM) route with supernatant (S), ultracentrifugation supernatant (Uₛ) or ultracentrifugation pellet (Uₚ) fractions from an in vitro culture of B. duncani (WA1 strain) in human erythrocytes. Quail-A was used as an adjuvant at (10 μg/dose) for each immunization. Un-immunized mice served as control. Blood samples were collected by retro-orbital bleeding from mice at the indicated timepoints, and sera were isolated to ascertain antibody titers. About 3 weeks after the 2^{nd} boost, mice were challenged with 10⁴ B. duncani-infected RBC by the intravenous (IV) route. Parasitemia profile and survival of the infected mice were monitored over 28 days post-infection.

FIG. 2 is a schematic representation of the isolation of vesicular fractions from in vitro culture of B. duncani-infected human erythrocytes, in accordance with some embodiments. B. duncani-infected red blood cells were maintained for 24 hours in serum-free media (DMEM-F12). Serum-free supernatant (Sup, S2) was collected by centrifugation at 18,000 RPM for 10 minutes at room temperature. Part of the S2 fraction was further subjected to centrifugation 16,000 g for 45 minutes and subsequently to ultracentrifugation at 120,000 g for 14 hours at 4° C. to collect the soluble (Us) and pellet (Up) fraction rich in vesicles. The ultracentrifugation supernatant (Uₛ) was collected and the resulting pellet (Uₚ) was resuspended in PBS with half the volume of the supernatant. 100 μL/mouse of each fraction was used for immunization (prime and the two subsequent boosts).

FIGS. 3A-3E demonstrate that immunizations with the supernatant (Sup) and ultracentrifugation pellet (U_{P}) fractions of FIGS. 1-2 elicited protective responses against B. duncani infection via injection of 10⁴ infected human red blood cells (iRBCs) in C3H/HeJ mice, in accordance with some embodiments. FIG. 3A: Control mice and mice immunized with the ultracentrifugation supernatant (U_{S}) fraction both have significantly reduced survival rates (zero in the case of the un-immunized control mice) after B. duncani challenge. In contrast, all the mice that were immunized with the S or the U_{P} fractions survived. FIG. 3B: Mice immunized with the supernatant (Sup) fraction were able to control the quantity of and ultimately eliminate the parasites in the blood, as measured by parasitemia. FIG. 3C: Mice immunized with the ultracentrifugation pellet (U_{P}) fraction were able to control the quantity of and ultimately eliminate the parasites in the blood, as measured by parasitemia. FIG. 3D: Some mice immunized with the ultracentrifugation supernatant (U_{S}) fraction cannot control the quantity of parasites in the blood (as measured by parasitemia) and died (see FIG. 3A). FIG. 3E: All un-immunized mice cannot control the quantity of parasites in the blood (as measured by parasitemia) and died (see FIG. 3A).

FIG. 4 shows the antibody profile of immunized mice to B. duncani proteins by indirect ELISA, in accordance with some embodiments. ELISA plates were coated with 100 uL of supernatant (S), Uₛ or Uₚ fractions, or 10 ng/well of purified B. duncani antigens (BdV3, BdV234, BdV235, BdV31, BdV36, BdV45, or BdV51). Sera from 5 mice in each group were collected on day 42 post-immunization, pooled and used at 1:5000 dilution in the ELISA assays. HRP conjugated anti-mouse secondary antibody was used to detect the antigen-antibody interaction. Absorbance was read at 450 nm in a SynergyMx plate reader.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

When residing in the red blood cells of the host, Babesia parasites produce large amounts of vesicles (also referred to as Babesia-derived vesicles or BDVs) that are then discharged into the host (Thekkiniath et al., Life Sci Alliance. 2019 Jun. 13; 2(3):e201900382). Similarly, Plasmodium parasites that cause malaria also release equivalent vesicles (Karam et al., EMBO Rep. 2022 Jul. 5;23(7):e54755. doi: 10.15252/embr.202254755). However, it is unclear as whether these vesicles and their components can be used as vaccines against the parasites (and possibly other pathogens).

In the study described herein (“the present study”), extracellular vesicles derived from an exemplary Babesia parasite, B. duncani, were tested as vaccines for human babesiosis in a mouse model. The extracellular vesicles derived from the parasite were demonstrated to be excellent vaccines against babesiosis, as the extracellular vesicles fully protected mice from B. duncani challenges, as judged by parasitemia and survival.

Apart from being able to confer strong protection against parasite infections, the vesicles are also easy to produce and can be generated in large scale under good manufacturing practice (GMP) conditions in vaccine programs.

It is worth-noting that donated red blood cells, when properly stored, have a shelf-life of 42 days. After 42 days in storage, unused red blood cells are considered “expired” and have to be discarded. Since the vesicles herein can be produced using “expired” red blood cells, the production of the vesicles herein is able to reduce unavoidable wastes exist in the blood donation system.

Furthermore, unlike many existing vaccines, the vesicles herein are stable and can be cryopreserved.

Accordingly, in some aspects, the present invention is directed to a composition suitable for immunizing a subject against parasite infections.

In some aspects, the present invention is directed to a method of immunizing a subject against an infection by a parasite.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

### Composition

In some aspects, the present invention is directed to a composition.

In some embodiments, the composition for immunizing a subject against a parasite infection, such as a vaccine composition.

In some embodiments, the composition is for parenteral such as intramuscular or intraperitoneal administration to a subject to establish an immunity against a parasite infection or to boost an existing immunity against a parasite infection.

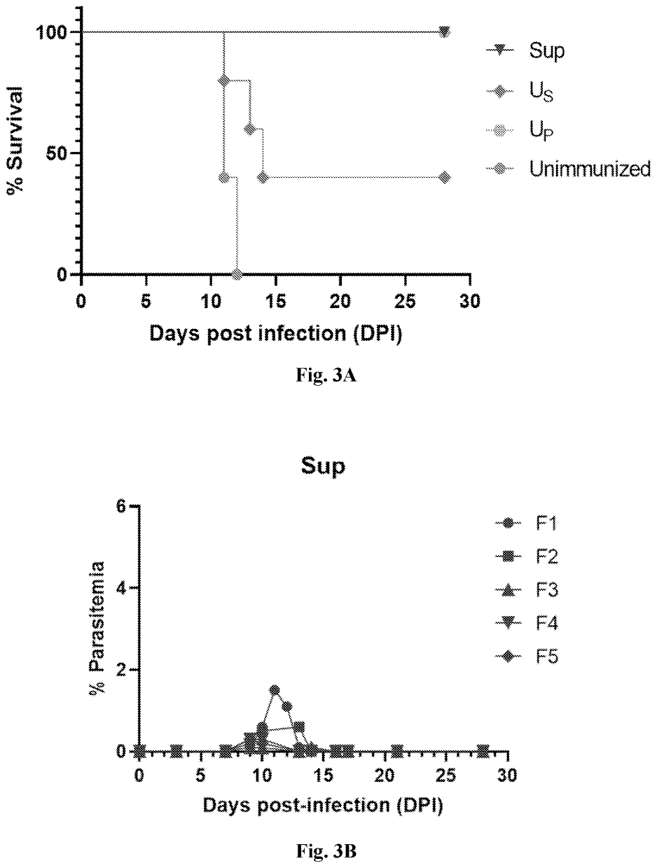

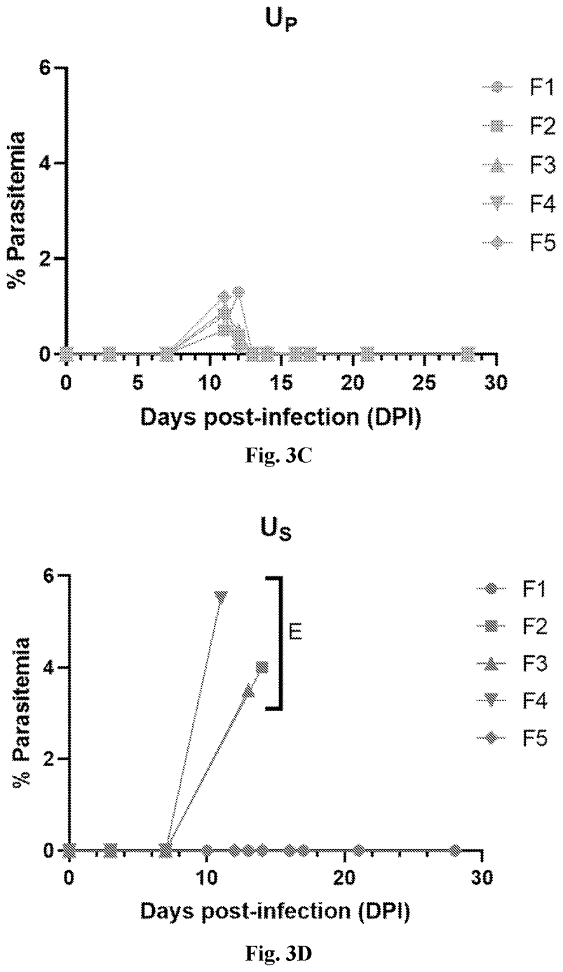

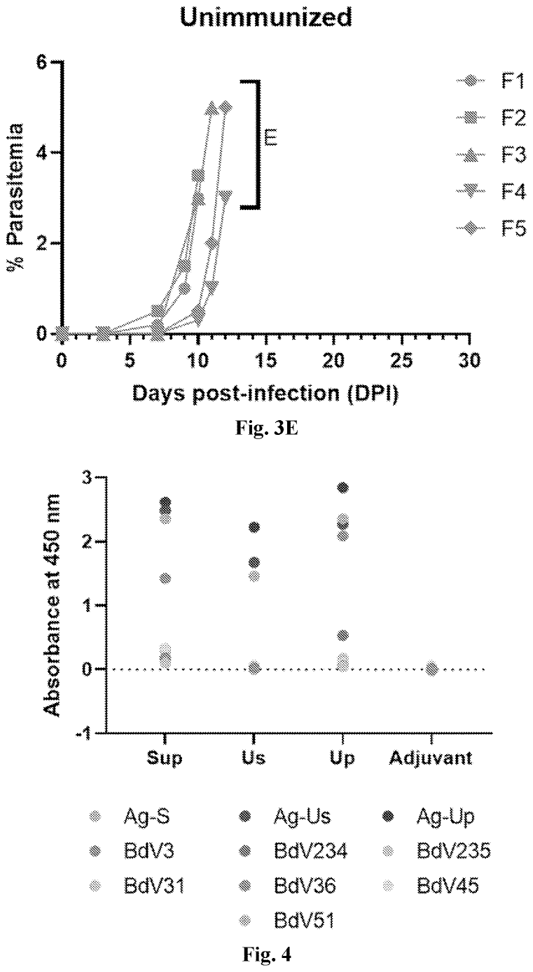

Babesia parasites and Plasmodium parasites are known to invade red blood cells of the host and release extracellular vesicles that contains parasite-derived proteins (see e.g., Thekkiniath et al., Life Sci Alliance. 2019 Jun. 13;2(3):e201900382 and Karam et al., EMBO Rep. 2022 Jul. 5;23(7):e54755. doi: 10.15252/embr.202254755). The study described herein, using the vesicles released in response to B. duncani infection as a non-limiting example, discovered that the extracellular vesicles released in response to parasite invasion, when used to immunizing animals, are able to confer strong immunity against the parasite (see e.g., FIGS. 3A-3C).

Accordingly, in some embodiments, the composition includes an extracellular vesicle produced by a red blood cell in response to being infected by a parasite.

In some embodiments, the composition further includes a pharmaceutically acceptable carrier for parenteral administration.

In some embodiments, parasite is a Babesia parasite.

In some embodiments, parasite is Babesia duncani or Babesia microti.

In some embodiments, the composition further includes an immunologic adjuvant.

In some embodiments, the immunologic adjuvant is an aluminum-based adjuvant, a lipid-based adjuvant, a nucleic acid-based adjuvant, a saponin-based adjuvant, or combinations thereof.

Non-limiting examples of aluminum-based adjuvants include amorphous aluminum hydroxyphosphate sulfate (AAHS), aluminum hydroxide, aluminum phosphate, potassium aluminum sulfate (Alum), or the like.

Non-limiting examples of lipid-based adjuvant include monophosphoryl lipid A (MPL) (an immune-boosting substance isolated from the surface of bacteria), QS-21 (a natural compound extracted from the Chilean soapbark tree Quillaja saponaria Molina), cholesterol, phospholipids, squalene, AS01_{B} (which includes MPL and QS-21), AS04 (which includes MPL and aluminum salts), MF59 (oil in water emulsion composed of squalene), and the like.

Non-limiting examples of nucleic acid-based adjuvant include CpG 1018 (cytosine phosphoguanine (CpG), a synthetic form of DNA that mimics bacterial and viral genetic material), and the like.

Non-limiting examples of saponin-based adjuvant includes Matrix-M™ (saponins derived from the soapbark tree Quillaja saponaria Molina).

In some embodiments, the extracellular vesicles herein are isolated by:
infecting red blood cells cultured in a culture medium with the parasite; and
centrifuging the culture medium at a centrifugation speed ranging from about 200 g to about 20,000 g (such as from about 250 g to about 10,000 g, from about 300 g to about 5,000 g, from about 350 g to about 1,000 g, or from about 400 g to about 500 g) and collecting at least a portion of the supernatant. In some embodiments, the extracellular vesicles are enriched in the collected supernatant.

In some embodiments, the extracellular vesicles are isolated by:
infecting red blood cells cultured in a culture medium with the parasite; and
centrifuging the culture medium at a centrifugation speed ranging from about 200 g to about 20,000 g (such as from about 250 g to about 10,000 g, from about 300 g to about 5,000 g, from about 350 g to about 1,000 g, or from about 400 g to about 500 g) and collecting at least a portion of the supernatant;
centrifuging the collected supernatant at a centrifugation speed of about 80,000 g or higher (such as about 90,000 g or higher, about 100,000 g or higher, about 120,000 g or higher, about 150,000 g or higher, about 175,000 g or higher, or about 200,000 g or higher) and collecting a pellet. In some embodiments, the extracellular vesicles are enriched in the collected pellet.

In some embodiments, instead of using ultracentrifugation (which sometimes have limited availability in parts of the world where the composition herein is needed), a low-centrifugation speed isolation technique is used instead. In some embodiments, the low-centrifugation speed isolation technique does not require centrifugation speed of 80,000 g or higher. Non-limiting examples of low-centrifugation speed isolation technique include nano-particle based methods, polymer-based methods (such as poly-ethylene glycol (PEG)-based precipitation), filtration based methods, immunoaffinity capture, microfluidic isolation, size-exclusion chromatography, and the like. Methods of isolating extracellular vesicles are described in, for example, Li et al. (Theranostics. 2017 Jan. 26;7(3):789-804) and Sidhom et al. (Int. J. Mol. Sci. 2020, 21(18), 6466).

In some embodiments, the extracellular vesicles are isolated using a polymer-based method for isolating extracellular vesicles. A non-limiting example of the polymer-based method is ExoQuick, which includes the following steps:
Infecting red blood cells cultured in a culture medium with the parasite;
Centrifuging the culture medium at a centrifugation speed ranging from 200 g to about 20,000 g to remove cellular debris and obtain a clarified biofluid;
Adding a polymer-based exosome extractor (such as ExoQuick or extractors of other extraction kit) to the clarified biofluid (for in ExoQuick, the ratio with the clarified biofluid of is about 1:4) and incubating the mixture (for about 30 minutes at 4° C.);
Centrifuging the extractor/biofluid mixture (at about 3,000 g for about 10 minute in the case of ExoQuick);
Removing supernatant (such as by aspiration; taking care to remove all residual solution without disturbing the precipitated extracellular vesicles); and
Re-suspending the pellets in a suitable amount of a resuspension buffer.

In some embodiments, the extracellular vesicles are enriched in the collected pellet.

The manual for ExoQuick can be found at www dot systembio dot com/wp/wp-content/uploads/2020/10/MANUAL_ExoQuick-ULTRA_Serum_Plasma_V3.pdf

### Method of Immunizing Against Parasite Infection

In some aspects, the present invention is directed to a method of immunizing a subject against a parasite infection.

In some embodiments, the method includes administering to the subject an extracellular vesicle produced by a red blood cell infected with the parasite.

In some embodiments, the parasite is a Babesia parasite.

In some embodiments, the parasite is Babesia duncani or Babesia microti.

In some embodiments, the subject is further administered with an immunologic adjuvant. In some embodiments, the immunologic adjuvant is the same as or similar to those as detailed elsewhere herein, such as in the “Composition” section.

In some embodiments, the extracellular vesicle herein is isolated by:
infecting the red blood cell cultured in a culture medium with the parasite; and
centrifuging the culture medium at a centrifugation speed ranging from about 200 g to about 20,000 g (such as from about 250 g to about 10,000 g, from about 300 g to about 5,000 g, from about 350 g to about 1,000 g, or from about 400 g to about 500 g) and collecting at least a portion of the supernatant. In some embodiments, the extracellular vesicle is enriched in the collected supernatant.

In some embodiments, the extracellular vesicle is isolated by:
infecting the red blood cell cultured in a culture medium with the parasite; and
centrifuging the culture medium at a centrifugation speed ranging from about 200 g to about 20,000 g (such as from about 250 g to about 10,000 g, from about 300 g to about 5,000 g, from about 350 g to about 1,000 g, or from about 400 g to about 500 g) and collecting at least a portion of the supernatant;
centrifuging the collected supernatant at a centrifugation speed of about 80,000 g or higher (such as about 90,000 g or higher, about 100,000 g or higher, about 120,000 g or higher, about 150,000 g or higher, about 175,000 g or higher, or about 200,000 g or higher) and collecting a pellet. In some embodiments, the extracellular vesicle is enriched in the collected pellet.

In some embodiments, the extracellular vesicle is isolated using a low-centrifugation speed isolation technique, which is described elsewhere herein.

In some embodiments, the subject is administered with a plurality of doses of the extracellular vesicle.

In some embodiments, the red blood cell for producing the extracellular vesicle is from the same species as that of the subject.

In some embodiments, the subject is a mammal, such as rodent (such as a mouse, a rat, a rabbit, or the like), an odd-toed ungulate (such as a horse, a donkey, or a mule), an even-toed ungulate (such as a cattle, a pig, a camelid, a sheep, a goat), a canine (such as a dog or a fox), a feline (such as a cat), primate (such as a monkey, a chimpanzee, a bonobo a gorilla, an orangutan, or a human), and the like. In some embodiments, the red blood cell for producing the extracellular vesicle is from infected red blood cells originated from animals of this paragraph.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1

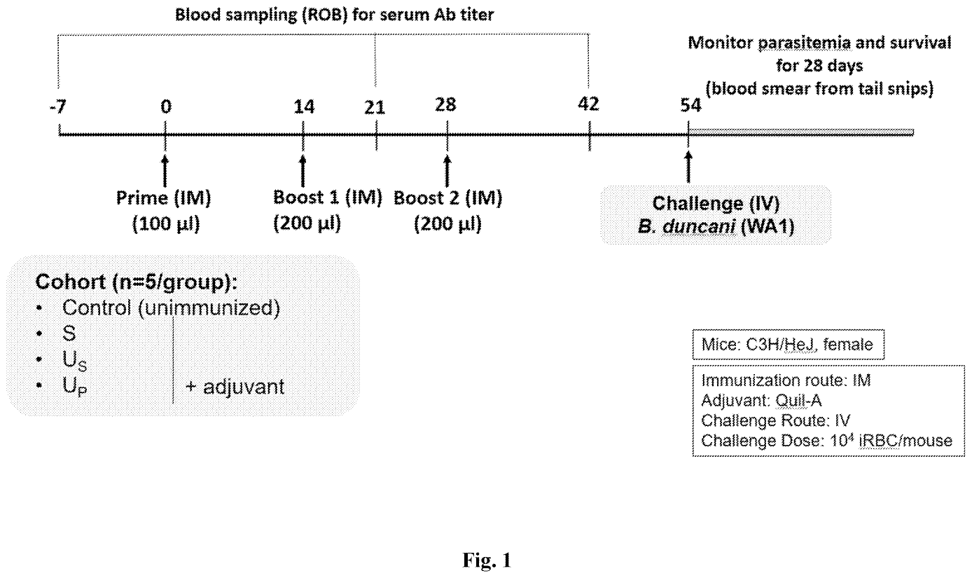

Referring to FIG. 1, in the present B. duncani vaccination study (“the present study”), female C3H/HeJ mice were either un-immunized (controls) or immunized with the supernatant (“S”), ultracentrifugation supernatant (“U_{S}”), or ultracentrifugation pellet (“U_{P}”) fraction of a B. duncani in vitro culture supernatant according to a prime and boost vaccination scheme. The immunized and control mice were then challenged with B. duncani pathogen intravenously at a dosage of 10⁴ iRBC/mouse. The parasitemia and survival of the mice were monitored for a period of 28 days.

### Example 2: Preparation of B. duncani in Vitro Culture Supernatant and Fractions Thereof

The culturing of B. duncani in vitro, as well as the isolation of extracellular vesicles produced in response to B. duncani infection, is performed in similar manners as those described in in Thekkiniath et al. (Life Sci Alliance. 2019 Jun. 13;2(3):e201900382. doi: 10.26508/lsa.201900382) and Baranyai et al. (PLoS One. 2015 Dec. 21;10(12):e0145686. doi: 10.1371/journal.pone.0145686. eCollection 2015). The entireties of both of the references are incorporated herein by reference.

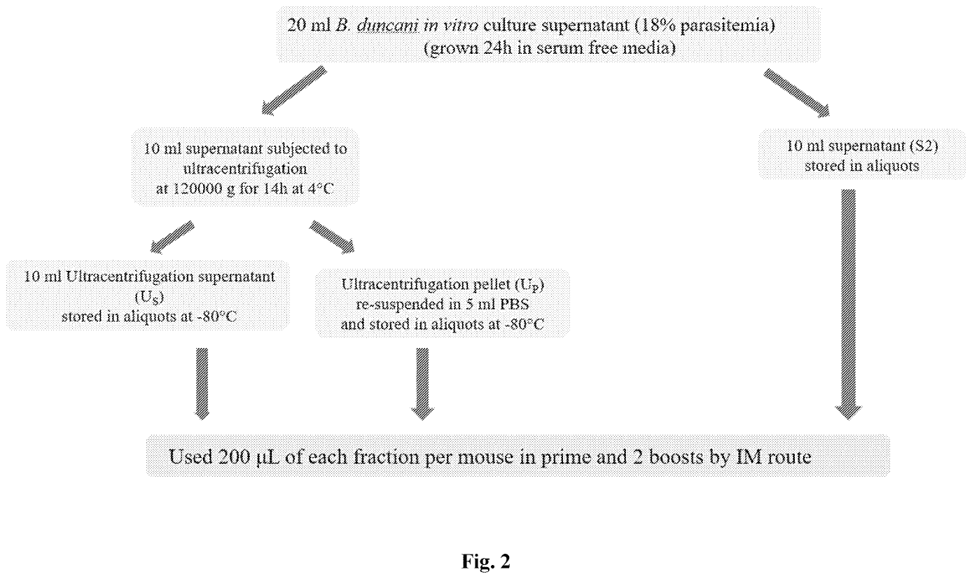

Briefly, referring to FIG. 2, 20 ml of B. duncani in vitro culture supernatant (18% parasitemia) (grown 24 h in serum free media) was divided into two equal portions. The first supernatant portion (10 ml) was used for further enrichment of the vesicles while the second supernatant portion (10 ml), which also contains the extracellular vesicles, was used for immunizing animals.

The first supernatant portion was subjected to ultracentrifugation at 120000 g for 14 h at 4° C. The resultant ultracentrifugation supernatant portion (U_{S}) is not expected to contain the extracellular vesicles and the ultracentrifugation pellet portion (U_{P}), which is expected to contain the extracellular vesicles, was re-suspended in 5 ml PBS and stored in aliquots at −80° C.

### Example 3: Immunization and Parasite Challenge

The second supernatant portion (S, which contains the extracellular vesicles), the ultracentrifugation supernatant portion (U_{S}, which does not contain the extracellular vesicles) and the ultracentrifugation pellet portion (U_{P}, which contains the extracellular vesicles) obtained in Example 2 were used to immunizing animals according to the vaccination scheme shown in FIG. 1 at days 0, 14 and 28. This is referred to herein as the “prime and boost” scheme.

At day 54, the animals were challenged with intravenous administration of B. duncani parasite at a dosage of 10⁴ B. duncani-infected RBCs/mouse. The animals were monitored for parasitemia and survival for 28 days afterwards.

### Example 4: Immunization With the Extracellular Vesicles Conferred Strong Protections Against Parasite Challenges

Referring to FIGS. 3A-3E, immunizations with the supernatant (Sup) and ultracentrifugation pellet (U_{P}) fractions, which contain the extracellular vesicles, elicited strong protective responses against 10⁴ B. duncani infection in C3H/HeJ mice.

Referring to FIG. 3A, control mice and mice immunized with the ultracentrifugation supernatant (U_{S}) fraction both have significantly reduced survival rates (zero in the case of the un-immunized control mice) in response to the B. duncani challenges. In contrast, all the mice that were immunized with the S or the U_{P} fractions (which contain the extracellular vesicles) survived.

Referring to FIGS. 3B and 3C, mice immunized with the supernatant (Sup) and the ultracentrifugation pellet (U_{P}) fractions (both of which contain the extracellular vesicles) were able to control the quantity of and ultimately eliminate the parasites in the blood, as measured by parasitemia.

Referring to FIGS. 3D and 3E, some mice immunized with the ultracentrifugation supernatant (U_{S}) fraction (which does not contain the extracellular vesicles) cannot control the quantity of parasites in the blood (as measured by parasitemia) and died (see FIG. 3A), and all un-immunized mice cannot control the quantity of parasites in the blood (as measured by parasitemia) and died (see FIG. 3A).

### Example 5: Humoral Response Elicited by Sup, U_{S}, U_{P} Immunizations

Referring to FIG. 4, Sup, U_{S}, and U_{P} immunizations elicited different levels of humoral responses. For example, immunizations with Sup and U_{P} fractions (both of which contains extracellular vesicles) includes stronger humoral responses against BdV3 than does the U_{S} fraction, which does not contain extracellular vesicles.

In FIG. 4, plates were coated with different B. duncani antigens indicated in FIG. 4 (10 ng/well for purified proteins or 200 μL/well for S, U_{S} or U_{P}), and d42 mouse sera were used at 1:5000 dilution.

### Enumerated Embodiments

In some aspects, the present invention is directed to the following non-limiting embodiments:

Embodiment 1: A composition, comprising: an extracellular vesicle produced by a red blood cell infected with a parasite; and a pharmaceutically acceptable carrier for parenteral administration.

Embodiment 2: The composition of Embodiment 1, wherein, when administered to a subject, the composition immunizes the subject against an infection of the parasite.

Embodiment 3: The composition of any one of Embodiments 1-2, wherein the parasite is one or more Babesia parasites.

Embodiment 4: The composition of any one of Embodiments 1-3, wherein the parasite is at least one selected from the group consisting of a B. duncani parasite, and a B. microti parasite.

Embodiment 5: The composition of any one of Embodiments 1-4, wherein the composition further comprises an immunologic adjuvant.

Embodiment 6: The composition of Embodiment 5, wherein the immunologic adjuvant is at least one selected from the group consisting of an aluminum-based adjuvant, a lipid-based adjuvant, a nucleic acid-based adjuvant, and a saponin-based adjuvant.

Embodiment 7: The composition of any one of Embodiments 1-6, wherein the extracellular vesicle is isolated by: infecting the red blood cell cultured in a culture medium with the parasite; and centrifuging the culture medium at a centrifugation speed ranging from about 200 g to about 20,000 g and collecting at least a portion of the supernatant, wherein the collected supernatant comprises the extracellular vesicle.

Embodiment 8: The composition of any one of Embodiments 1-6, wherein the extracellular vesicle is isolated by: infecting the red blood cell cultured in a culture medium with the parasite; centrifuging the culture medium at a centrifugation speed ranging from about 200 g to about 20,000 g and collecting at least a portion of the supernatant; and centrifuging the supernatant at a centrifugation speed of about 80,000 g or higher so as to collect a pellet, wherein the collected pellet comprises the extracellular vesicle.

Embodiment 9: The composition of any one of Embodiments 1-8, wherein the extracellular vesicle is isolated by low-centrifugation speed isolation technique, such as a nano-particle based method, a polymer-based method, a filtration-based method, an immunoaffinity capture, a microfluidic isolation, a or size-exclusion chromatography.

Embodiment 10: A method of immunizing a subject against an infection by a parasite, comprising: administering to the subject an extracellular vesicle produced by a red blood cell infected with the parasite.

Embodiment 11: The method of Embodiment 10, wherein the parasite is one or more Babesia parasites.

Embodiment 12: The method of any one of Embodiments 10-11, wherein the parasite is at least one selected from the group consisting of a B. duncani parasite and a B. microti parasite.

Embodiment 13: The method of any one of Embodiments 10-12, wherein the subject is further administered with an immunologic adjuvant.

Embodiment 14: The method of Embodiment 13, wherein the immunologic adjuvant is at least one selected from the group consisting of an aluminum-based adjuvant, a lipid-based adjuvant, a nucleic acid-based adjuvant, and a saponin-based adjuvant.

Embodiment 15: The method of any one of Embodiments 10-14, wherein the extracellular vesicle is an isolated extracellular vesicle, and wherein the extracellular vesicle is isolated by: infecting the red blood cell cultured in a culture medium with the parasite; and centrifuging the culture medium at a centrifugation speed ranging from about 200 g to about 20,000 g and collecting at least a portion of the supernatant, wherein the collected supernatant comprises the extracellular vesicle.

Embodiment 16: The method of any one of Embodiments 10-14, wherein the extracellular vesicle is an isolated extracellular vesicle, and wherein the extracellular vesicle is isolated by: infecting the red blood cell cultured in a culture medium with the parasite;
centrifuging the culture medium at a centrifugation speed ranging from about 200 g to about 20,000 g so and collecting at least a portion of the supernatant; and centrifuging the supernatant at a centrifugation speed of about 80,000 g or higher so as to collect a pellet, wherein the collected pellet comprises the extracellular vesicle.

Embodiment 17: The method of any one of Embodiments 10-16, wherein the subject is administered with a plurality of doses of the extracellular vesicle.

Embodiment 18: The method of any one of Embodiments 10-17, wherein the red blood cell is from the same species as that of the subject.

Embodiment 19: The method of any one of Embodiments 10-18, wherein the subject is a mammal.

Embodiment 20: The method of any one of Embodiments 10-19, wherein the subject is a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A composition, comprising:
an extracellular vesicle produced by a red blood cell infected with a parasite; and
a pharmaceutically acceptable carrier for parenteral administration.

2. The composition of claim 1, wherein, when administered to a subject, the composition immunizes the subject against an infection of the parasite.

3. The composition of claim 1, wherein the parasite is one or more Babesia parasites.

4. The composition of claim 1, wherein the parasite is at least one selected from the group consisting of a B. duncani parasite and a B. microti parasite.

5. The composition of claim 1, wherein the composition further comprises an immunologic adjuvant.

6. The composition of claim 5, wherein the immunologic adjuvant is at least one selected from the group consisting of an aluminum-based adjuvant, a lipid-based adjuvant, a nucleic acid-based adjuvant, and a saponin-based adjuvant.

7. The composition of claim 1, wherein the extracellular vesicle is isolated by:
infecting the red blood cell cultured in a culture medium with the parasite; and
centrifuging the culture medium at a centrifugation speed ranging from about 200 g to about 20,000 g and collecting at least a portion of the supernatant,
wherein the collected supernatant comprises the extracellular vesicle.

8. The composition of claim 1, wherein the extracellular vesicle is isolated by:
infecting the red blood cell cultured in a culture medium with the parasite;
centrifuging the culture medium at a centrifugation speed ranging from about 200 g to about 20,000 g and collecting at least a portion of the supernatant; and
centrifuging the supernatant at a centrifugation speed of about 80,000 g or higher so as to collect a pellet,
wherein the collected pellet comprises the extracellular vesicle.

9. The composition of claim 1, wherein the extracellular vesicle is isolated by low-centrifugation speed isolation technique, such as a nano-particle based method, a polymer-based method, a filtration-based method, an immunoaffinity capture, a microfluidic isolation, a or size-exclusion chromatography.

10. A method of immunizing a subject against an infection by a parasite, comprising:
administering to the subject an extracellular vesicle produced by a red blood cell infected with the parasite.

11. The method of claim 10, wherein the parasite is one or more Babesia parasites.

12. The method of claim 10, wherein the parasite is at least one selected from the group consisting of a B. duncani parasite and a B. microti parasite.

13. The method of claim 10, wherein the subject is further administered with an immunologic adjuvant.

14. The method of claim 13, wherein the immunologic adjuvant is at least one selected from the group consisting of an aluminum-based adjuvant, a lipid-based adjuvant, a nucleic acid-based adjuvant, and a saponin-based adjuvant.

15. The method of claim 10, wherein the extracellular vesicle is an isolated extracellular vesicle, and wherein the extracellular vesicle is isolated by:
infecting the red blood cell cultured in a culture medium with the parasite; and
centrifuging the culture medium at a centrifugation speed ranging from about 200 g to about 20,000 g and collecting at least a portion of the supernatant,
wherein the collected supernatant comprises the extracellular vesicle.

16. The method of claim 10, wherein the extracellular vesicle is an isolated extracellular vesicle, and wherein the extracellular vesicle is isolated by:
infecting the red blood cell cultured in a culture medium with the parasite; and
centrifuging the culture medium at a centrifugation speed ranging from about 200 g to about 20,000 g so and collecting at least a portion of the supernatant;
centrifuging the supernatant at a centrifugation speed of about 80,000 g or higher so as to collect a pellet,
wherein the collected pellet comprises the extracellular vesicle.

17. The method of claim 10, wherein the subject is administered with a plurality of doses of the extracellular vesicle.

18. The method of claim 10, wherein the red blood cell is from the same species as that of the subject.

19. The method of claim 10, wherein the subject is a mammal.

20. The method of claim 10, wherein the subject is a human.
